(19) United States
(12) Patent Application Publication
(10) Pub. No.: US 2025/0336634 A1
(43) Pub. Date: Oct. 30, 2025

(54) X-RAY IMAGING APPARATUS AND X-RAY IMAGING METHOD

(71) Applicant: SHIMADZU CORPORATION, Kyoto-shi (JP)

(72) Inventors: Akira HORIBA, Kyoto-shi (JP); Kenji KIMURA, Kyoto-shi (JP); Tomomi TAMURA, Kyoto-shi (JP); Yukihisa WADA, Kyoto-shi (JP); Satoshi SANO, Kyoto-shi (JP)

(21) Appl. No.: 18/726,420

(22) PCT Filed: Jan. 5, 2022
(86) PCT No.: PCT/JP2022/000137

§ 371 Date: Mar. 26, 2025

(51) Int. Cl. H01J 35/14; G01N 23/046; H01J 35/06

(52) U.S. Cl. CPC H01J 35/147; G01N 23/046; H01J 35/065; G01N 2223/3306; G01N 2223/419

(57) ABSTRACT

This X-ray imaging apparatus (100) is equipped with an imaging control unit (6) that controls an X-ray source (1) so that X-ray irradiation is performed by a subset of electron emission units (12) selected from a plurality of electron emission units (12), for each imaging angle (40) when acquiring a plurality of projection image data (50), and also control a selection of a second electron emission unit (42) different from a first electron emission unit (41) used in immediately preceding X-ray irradiation when performing X-ray irradiation.

### TECHNICAL FIELD

The present invention relates to an X-ray imaging apparatus and an X-ray imaging method, and more particularly, an X-ray imaging apparatus and an X-ray imaging method for performing CT (Computed Tomography) imaging of a subject.

### BACKGROUND ART

Conventionally, an X-ray imaging apparatus that performs CT imaging of a subject is known. Such an X-ray imaging apparatus is disclosed, for example, in Japanese Unexamined Patent Application Publication No. 2018-46976.

In Japanese Unexamined Patent Application Publication No. 2018-46976, an X-ray imaging apparatus equipped with an X-ray tube, a flat panel detector, a stage for placing a subject thereon, and an image reconstruction unit is disclosed. The stage rotates with the subject placed on it. The X-ray tube emits X-rays toward the subject that rotates along with the stage. The flat panel detector detects X-rays emitted from the X-ray tube and transmitted through the subject. The image reconstruction unit reconstructs an image using the iterative method based on the X-ray projection data acquired by the flat panel detector.

### PRIOR ART DOCUMENT

### Patent Document

Patent Document 1: Japanese Unexamined Patent Application Publication No. 2018-46976

### SUMMARY OF THE INVENTION

### Problems to be Solved by the Invention

Although not disclosed in Japanese Unexamined Patent Application Publication No. 2018-46976, the X-ray tube is equipped with an electron source and a target that generates X-rays when an electron beam emitted from the electron source collides with it. In order to increase the spatial resolution of a CT image, it has been desired to reduce the focal spot size of X-rays formed by an electron beam.

However, the smaller the focal spot size of X-rays, the more likely the target is to be damaged by the heat generated when the electron beam strikes a narrow area of the target, causing the local area of the target to heat up intensively. Particularly in the case of capturing a CT image, the X-ray irradiation time becomes longer since the subject is imaged from various imaging angles, and therefore, the target is easily damaged. For this reason, it has been desired that the damage to the target be suppressed even when the focal spot size of X-rays is reduced.

The present invention has been made to solve the above-described problems, and one object of the present invention is to provide an X-ray imaging apparatus and an X-ray imaging method capable of suppressing damage to a target even when the focal spot size of X-rays is reduced.

### Means for Solving the Problems

In order to attain the above-described object, an X-ray imaging apparatus according to a first aspect of the present invention comprises:
an X-ray source including a target and a plurality of electron emission units, each of the plurality of electron emission units being configured to emit electrons to a different focal position on the target such that electron beam axes extending from the plurality of electron emission units to the target do not intersect with each other;
a detector configured to detect X-rays emitted from the X-ray source;
a subject mounting unit arranged between the X-ray source and the detector to support a subject;
a rotation mechanism configured to relatively rotate an imaging unit and the subject mounting unit to change an imaging angle of the subject, the imaging unit including the X-ray source and the detector;
an image processing unit configured to acquire a plurality of projection image data, one at each of a plurality of imaging angles, from the detector, and to generate a CT image based on an acquired plurality of projection image data; and
an imaging control unit configured to control the X-ray source so that X-ray irradiation is performed by a subset of electron emission units selected from the plurality of electron emission units, for each imaging angle when acquiring the plurality of projection image data, and to control a selection of a second electron emission unit different from a first electron emission unit used in immediately preceding X-ray irradiation when performing X-ray irradiation.

An X-ray imaging method according to a second aspect of the present invention comprises:
a first step of performing X-ray irradiation by a subset of electron emission units selected from a plurality of electron emission units, from an X-ray source that includes a target and the plurality of electron emission units, the X-ray source being configured to emit electrons to different focal positions on the target such that electron beam axes extending from the plurality of electron emission units to the target do not intersect with each other;
a second step of acquiring projection image data by detecting X-rays emitted from the X-ray source and transmitted through a subject by a detector;
a third step of changing an imaging angle of the subject by relatively rotating the X-ray source and the detector and the subject;
a fourth step of selecting a second electron emission unit out of the plurality of electron emission units, the second electron emission unit being different from a first electron emission unit used for immediately preceding X-ray irradiation;
a step of acquiring a plurality of projection image data, one at each of a plurality of imaging angles, by repeating the first step to the fourth step; and
a step of generating a CT image based on the acquired plurality of projection image data.

Note that in the present invention, “a subset of electron emission units” refers to one or more of a plurality of electron emission units and a smaller number of electron emission units than the total number of the plurality of electron emission units, which are selected for use in X-ray irradiation. The “first electron emission unit” refers to “a subset of electron emission units” selected in the immediately preceding X-ray irradiation among the plurality of electron emission units. The “second electron emission unit” refers to the “subset of electron emission units” selected in the X-ray irradiation subsequent to the irradiation by the “first electron emission unit” among the plurality of electron emission units.

### Effects of the Invention

In the X-ray imaging apparatus according to the first aspect, as described above, it is provided with
an X-ray source including a target and a plurality of electron emission units, each of the plurality of electron emission units being configured to emit electrons to a different focal position on the target such that electron beam axes extending from the plurality of electron emission units to the target do not intersect with each other, and
an imaging control unit configured to control the X-ray source so that X-ray irradiation is performed by a subset of electron emission units selected from the plurality of electron emission units, for each imaging angle when acquiring the plurality of projection image data, and to control a selection of a second electron emission unit different from a first electron emission unit used in immediately preceding X-ray irradiation when performing X-ray irradiation.

With this, when sequentially performing X-ray irradiation for each imaging angle, the focal position on the target in the immediately preceding X-ray irradiation by the first electron emission unit and the focal position on the target in the subsequent X-ray irradiation by the second electron emission unit can be made different. Therefore, even if heat is generated intensively at the focal position by reducing the focal spot size of X-rays, the focal position on the target is changed each time X-ray irradiation is performed, allowing the heat-generating points on the target to be distributed. As a result, compared with the case in which the same focal position of the target continuously heats up, the local temperature rise on the target can be reduced, thereby allowing damage to the target to be suppressed even if the focal spot size of X-rays is reduced.

In the X-ray imaging method according to the second aspect, as described above, the method comprises:
a first step of performing X-ray irradiation by a subset of electron emission units selected from a plurality of electron emission units, from an X-ray source that includes a target and the plurality of electron emission units, the X-ray source being configured to emit electrons to different focal positions on the target such that electron beam axes extending from the plurality of electron emission units to the target do not intersect with each other, and
a fourth step of selecting a second electron emission unit out of the plurality of electron emission units, the second electron emission unit being different from a first electron emission unit used for immediately preceding X-ray irradiation.

With this, when sequentially performing X-ray irradiation for each imaging angle, it is possible to differentiate between the focal position on the target in the immediately preceding X-ray irradiation by the first electron emission unit and the focal position on the target in the subsequent X-ray irradiation by the second electron emission unit. Therefore, even if heat is generated intensively at the focal spot by reducing the focal spot size of X-rays, the focal position on the target is changed each time X-ray irradiation is performed, allowing the heat-generating points on the target to be distributed. As a result, compared with the case where the same focal position of the target continuously heats up, the local temperature rise on the target can be reduced, thereby allowing damage to the target to be suppressed even if the focal spot size of X-rays is reduced.

### BRIEF DESCRIPTION OF THE DRAWINGS

FIG. 1 is a schematic diagram showing the entire configuration of an X-ray imaging apparatus according to one embodiment.

FIG. 2 is a diagram for explaining the configuration in which one of a plurality of electron emission units in an X-ray source is selected for imaging.

FIG. 3 is a schematic diagram for explaining the configuration of a plurality of electron emission units.

FIG. 4 is a schematic diagram for explaining the configuration of a cold cathode electron source included in an electron emission unit.

FIG. 5 is a schematic diagram for explaining a plurality of focal positions on a target.

FIG. 6 is a first diagram for explaining selection of an electron emission unit when performing imaging.

FIG. 7 is a second diagram for explaining selection of an electron emission unit when performing imaging.

FIG. 8 is a third diagram for explaining selection of an electron emission unit when performing imaging.

FIG. 9 is a diagram showing various data stored in a storage unit.

FIG. 10 is a schematic diagram for explaining a spatial coordinate system in the reconstruction process.

FIG. 11 is a first schematic diagram for explaining the reconstruction process.

FIG. 12 is a second diagram for explaining the reconstruction process.

FIG. 13 is a flowchart for explaining the imaging operation of the X-ray imaging apparatus.

FIG. 14 is a flowchart for explaining the reconstruction process flow.

FIG. 15 is a schematic diagram showing a modification of the X-ray imaging apparatus.

### EMBODIMENTS FOR CARRYING OUT THE INVENTION

Hereinafter, some embodiments in which the present invention is embodied will be described based on the attached drawings.

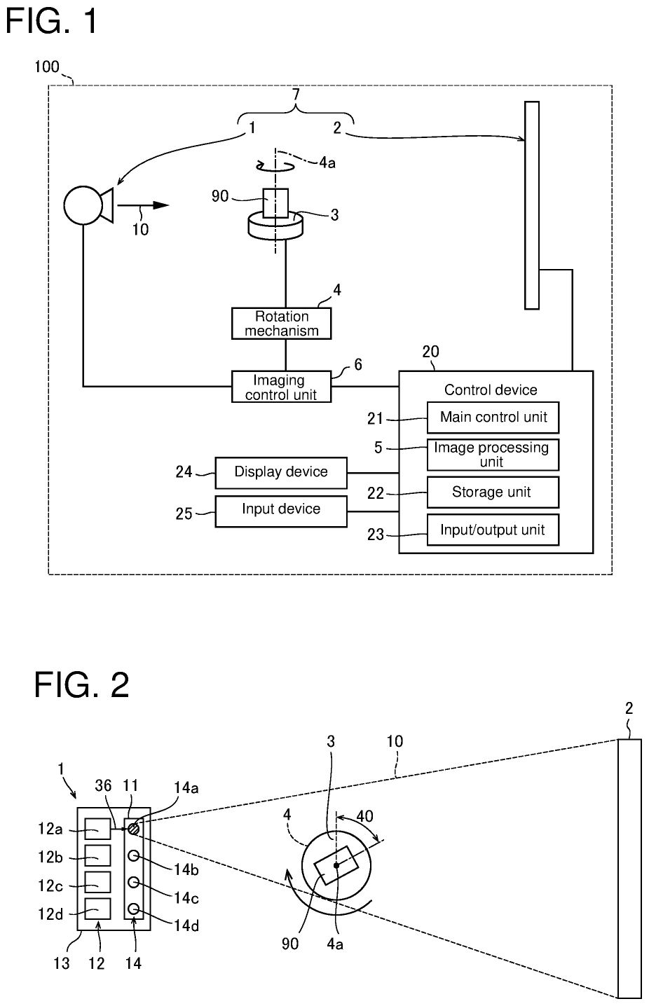

First, referring to FIG. 1, the entire configuration of the X-ray imaging apparatus 100 according to one embodiment will be described.

As shown in FIG. 1, the X-ray imaging apparatus 100 is an apparatus for capturing an X-ray CT image of a subject 90. The X-ray imaging apparatus 100 of this embodiment is used for, e.g., non-destructive testing applications. In this case, the subject 90 is a sample to be inspected.

The X-ray imaging apparatus 100 is equipped with an X-ray source 1, a detector 2, a subject mounting unit 3, a rotation mechanism 4, an image processing unit 5, and an imaging control unit 6. The X-ray source 1 and the detector 2 constitute an imaging unit 7 that captures X-ray images.

The X-ray source 1 is configured to irradiate the subject 90 placed on the subject mounting unit 3 with X-rays 10. The X-ray source 1 is configured to generate X-rays 10 when a high voltage is applied. The X-ray source 1 faces the detector 2 via the subject mounting unit 3. In this embodiment, the X-ray source 1, the subject mounting unit 3, and the detector 2 are arranged side by side in the horizontal direction.

The detector 2 is configured to detect the X-rays emitted from the X-ray source 1. The X-rays 10 emitted from the X-ray source 1 pass through the subject 90 and enter the detection surface of the detector 2. The detector 2 is configured to convert the detected X-rays 10 into an electrical signal. This produces an X-ray image that reflects the transmission of the X-rays 10 through the subject 90. The detector 2 is, for example, an FPD (Flat Panel Detector). The detector 2 is composed of a plurality of conversion elements (not illustrated) and pixel electrodes (not illustrated) arranged on the plurality of conversion elements. The plurality of conversion elements and pixel electrodes are arranged in a matrix-like pattern on the detection plane at predetermined intervals (pixel pitches). The detection signal (image signal) from the detector 2 is sent to image processing unit 5.

The subject mounting unit 3 is positioned between the X-ray source 1 and the detector 2 and is configured to support the subject 90. In this embodiment, the subject mounting unit 3 is constituted by a subject stage on which the subject 90 is mounted. In some cases, the subject 90 is mounted on the subject mounting unit 3 via a holder (not illustrated) or other means that holds the subject 90.

The rotation mechanism 4 relatively rotates the imaging unit 7, which includes the X-ray source 1 and detector 2, and the subject mounting unit 3. With this, the rotation mechanism 4 is configured to change the imaging angle 40 of the subject 90. The rotation mechanism 4 relatively rotates the imaging unit 7 and the subject mounting unit 3 about the rotation axis 4a. The rotation axis 4a is orthogonal to the straight line (representative line of the X-ray flux) extending from the X-ray source 1 to the detector 2 through the subject 90 on the subject mounting unit 3. In this embodiment, the rotation axis 4a passes through the subject mounting unit 3 and extends along the vertical direction.

The rotation mechanism 4 rotates either one of or both of the imaging unit 7 and the subject mounting unit 3 about the rotation axis 4a. In this embodiment, the rotation mechanism 4 rotates the subject mounting unit 3 about the rotation axis 4a within the horizontal plane. The rotation mechanism 4 does not rotate the imaging unit 7. The rotation mechanism 4 includes a motor (not illustrated) for rotating the subject mounting unit 3, which is the object stage, and a reduction gear (not illustrated). In this embodiment, the subject mounting unit 3 and the rotation mechanism 4 constitute the rotation stage for the subject 90.

In accordance with the rotation of the subject mounting unit 3, the subject 90 supported by the subject mounting unit 3 is rotated about the rotation axis 4a within the horizontal plane. As the subject mounting unit 3 rotates, the imaging angle 40 (see FIG. 2) of the subject 90 changes. The imaging angle is a relative angle between the subject 90 and the imaging unit 7. In this embodiment, the imaging angle 40 is the angle of the subject mounting unit 3 about the rotation axis 4a, with the origin angle (initial angle) of the rotation mechanism 4 as 0 degrees. FIG. 2 shows an example of the subject mounting unit 3 rotated from the origin angle to a certain imaging angle 40. The rotation mechanism 4 can rotate the subject mounting unit 3 to any angle so that the subject 90 is positioned at any imaging angle 40.

Returning to FIG. 1, the image processing unit 5 is provided in the control device 20. The control device 20 is configured, for example, by a personal computer (PC). The control device 20 is equipped with a main control unit 21, an image processing unit 5, a storage unit 22, and an input/output unit 23. The control device 20 is connected to a display device 24 and an input device 25.

The main control unit 21 is composed of, for example, a processor such as a CPU (Central Processing Unit), and performs setting of imaging conditions and control of the start and stop of imaging in the X-ray imaging apparatus 100 by executing application programs stored in the storage unit 22.

The image processing unit 5 is a processor, such as, e.g., a GPU (Graphics Processing Unit) or an FPGA (Field-Programmable Gate Array) configured for image processing.

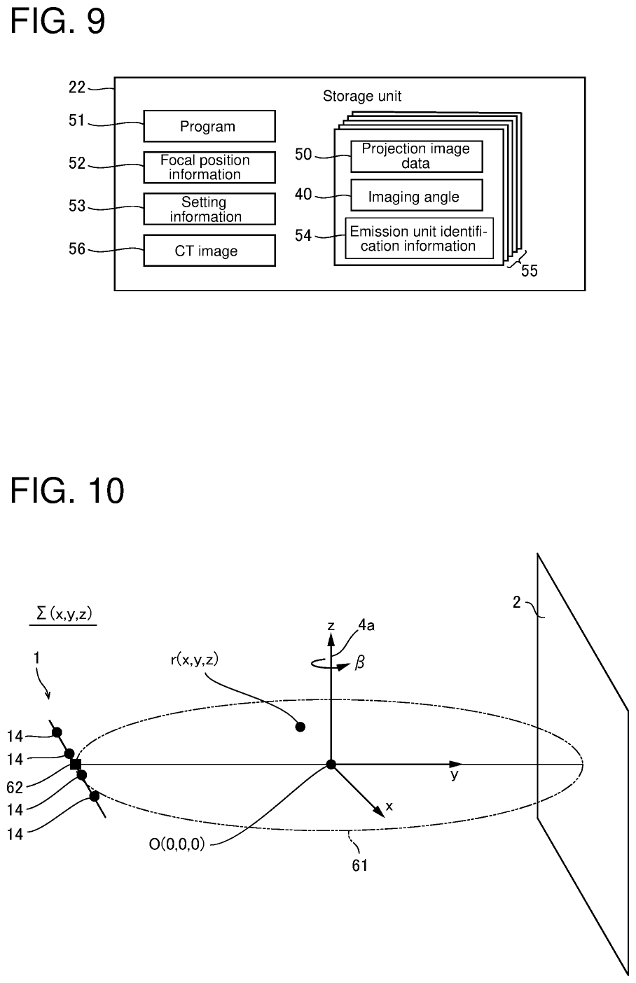

The image processing unit 5 acquires a plurality of projection image data 50 (see FIG. 9) at each of the plurality of imaging angles 40 from the detector 2. In other words, the image processing unit 5 generates projection image data 50 from the detection signal (image signal) of the detector 2 for each of the imaging angles 40. As described above, by changing the imaging angle 40 of the subject 90 by the rotation mechanism 4, X-ray images of the subject 90 are captured by the imaging unit 7 at each of the plurality of preset imaging angles 40. The projection image data 50 is data of an X-ray image acquired for each of the imaging angles 40.

The acquisition of projection image data 50 for each of the imaging angles 40 is performed over a predetermined angular range. The predetermined angular range is 360 degrees (one rotation). Further, the projection image data 50 is acquired by the number of projection image data corresponding to the predetermined number of imaging angles (number of views). In this embodiment, the plurality of imaging angles 40 is each of the angles set at equal angular intervals, with 360 degrees (one rotation) divided by the number of imaging angles. Therefore, the plurality of projection image data 50 is an X-ray image acquired at each of the imaging angles 40, in which the imaging unit 7 and the subject 90 are rotated relative to each other sequentially by a unit angle corresponding to the number of imaging angles.

The image processing unit 5 is configured to generate a CT image 56 (see FIG. 9) based on the acquired plurality of projection image data 50 (see FIG. 9). The image processing unit 5 generates a CT image 56 by performing a reconstruction process on a set (which will be referred to as “projection data set”) of a plurality of projection image data 50 for each of the imaging angles 40 over 360 degrees. The CT image 56 is an image that reflects the three-dimensional structure of the subject 90, and is reconstructed by arithmetic processing from a plurality of X-ray images (projection image data 50) captured at various imaging angles 40. The CT image 56 can be in the form of a tomographic image of the subject 90, a three-dimensional stereoscopic image, etc.

The storage unit 22 is configured to include a volatile storage unit and a nonvolatile storage unit. The storage unit 22 stores the program 51 (see FIG. 9), various setting information 53 (see FIG. 9) and other information related to CT imaging of the X-ray imaging apparatus 100. The storage unit 22 stores the plurality of acquired projection image data 50 (see FIG. 9) and the CT image 56 generated based on those projection image data 50.

The input/output unit 23 is configured by various interfaces for inputting/outputting signals to/from the control device 20. The input/output unit 23 is connected to the display device 24 and an input device 25. The display device 24 is, for example, a liquid crystal display. The input device 25 includes a keyboard and a mouse. The image processing unit 5 acquires a detection signal (image signal) from the detector 2 via the input/output unit 23. The main control unit 21 transmits an instruction, such as an instruction to start or stop imaging, to the imaging control unit 6 via the input/output unit 23.

The imaging control unit 6 controls the operation of the X-ray source 1. Further, the imaging control unit 6 controls the operation of the rotation mechanism 4. The imaging control unit 6 is configured by, e.g., a control device for the X-ray source 1 and a control device for the rotation mechanism 4. The imaging control unit 6 performs control to emit X-rays 10 from the X-ray source 1 and to stop the emission when acquiring a plurality of projection image data 50 (see FIG. 9), and also controls the rotation mechanism 4 so that the subject 90 is sequentially positioned at the plurality of imaging angles 40.

### Configuration of X-ray Source

As shown in FIG. 2, in this embodiment, the X-ray source 1 includes a target 11 and a plurality of electron emission units 12. The target 11 and the plurality of electron emission units 12 are housed in a vacuum container 13.

The X-ray source 1 is configured to emit electrons from the electron emission unit 12 by applying a voltage between the electron emission unit 12 as a cathode and the target 11 as an anode, and to generate X-rays 10 from the target 11 by colliding the emitted electrons against the target 11.

The plurality of electron emission units 12 is configured to emit electrons to different focal positions 14 on the target 11 such that the electron beam axes extending from each of the plurality of electron emission units 12 to the target 11 do not intersect each other. The imaging control unit 6 in FIG. 1 can control the electron emission from the plurality of electron emission units 12 individually. The imaging control unit 6 can select any one of the plurality of electron emission units 12 to emit an electron beam 36, and can simultaneously emit the electron beam 36 from the selected plurality of electron emission units 12 or all of the electron emission units 12. For this reason, in this embodiment, the X-ray source 1 can emit X-rays 10 from mutually different focal points (focal positions 14), the number of which is equal to the number of electron emission units 12 equipped by the X-ray source 1.

The number of the electron emission units 12 is not specifically limited. The number of electron emission units 12 provided in the X-ray source 1 can be, for example, 2, 3, 4, 5, 10, 20, 50, or 100. In FIG. 2, only the four electron emission units 12a, 12b, 12c, and 12d are illustrated for convenience. The four electron emission units 12a, 12b, 12c, and 12d are capable of emitting an electron beam 36 toward the respective focal positions 14a, 14b, 14c, and 14d on the target 11. With this, it is possible to emit X-rays 10 from the focal positions 14a, 14b, 14c, and 14d corresponding to the electron emission units 12a, 12b, 12c, and 12d, respectively, toward the detector 2.

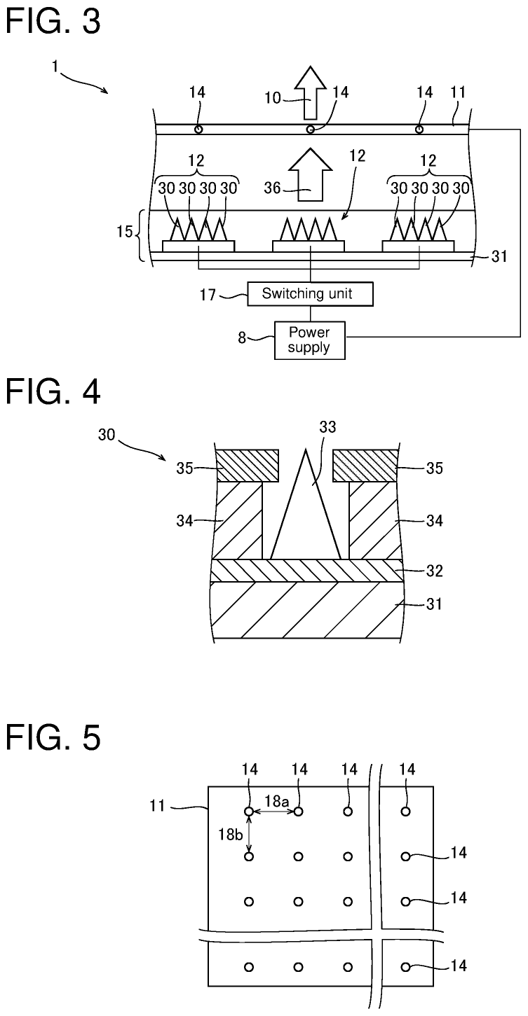

The structure of the target 11 is not specifically limited. The target 11 may be either of the reflective type (not shown) or of the transmissive type (see FIG. 3). A reflective type target is a target of the type that has a surface inclined at an angle with respect to the electron beam 36 and emits X-rays 10 such that the X-rays are reflected by the inclined surface in a direction different from the traveling direction of the electron beam 36. A transmission type target is a target of the type that has a pair of surfaces (front and back surfaces) orthogonal to the electron beam 36, and emits X-rays 10 from the other surface such that the impact of the electron beam 36 on one surface causes the X-rays 10 to pass through the target. Further, the target 11 may be fixed in the vacuum container 13 in a stationary state, or may be rotated by a driving source, such as a motor. In other words, the X-ray source 1 may have a so-called rotating anode structure.

FIG. 3 shows a more detailed configuration of the electron emission unit 12 and the target 11. FIG. 3 shows an example of a transmissive type target. In FIG. 3, the X-ray source 1 includes an electron source unit 15 with a plurality of cold cathode electron sources 30 arranged on a plane. The plurality of electron emission units 12 is each configured by different groups composed of a plurality of cold cathode electron sources 30.

The electron source unit 15 is formed such that numerous cold cathode electron sources 30 are arrayed on a substrate 31 by applying semiconductor manufacturing technology. The substrate 31 is a flat plate made of a material, such as silicon and glass. A group composed of some of the plurality of cold cathode electron sources 30 arranged in an array constitutes one electron emission unit 12.

The group that constitutes one of the plurality of electron emission units 12 is composed of one or more cold cathode electron sources 30 that emit electrons to the same focal position 14 on the target 11. One electron emission unit 12 includes one or more cold cathode electron sources 30. One electron emission unit 12 includes ten or more, or 1,000 or more of cold cathode electron sources 30. In the case where one electron emission unit 12 is composed of a plurality of cold cathode electron sources 30, the set of electrons emitted from each of the plurality of cold cathode electron sources 30 constituting the electron emission unit 12 forms the electron beam 36 emitted from the electron emission unit 12. The electron beam 36 is directed toward one focal position 14 on the target 11. The collision of the electron beam 36 causes X-rays 10 to be generated from the focal position 14 on the target 11. The spot (point region) where the electron beam 36 collides at the focal position 14 serves as the focus of the X-rays 10.

The individual cold cathode electron source 30 is a field emission type electron source that emits electrons from an emitter to which an electric field is applied by the tunneling effect. The cold cathode electron source 30 is, for example, a Spindt-type electron source, as shown in FIG. 4. The Spindt-type electron source includes a cathode electrode 32 formed on a substrate 31, a tapered emitter 33 formed on the cathode electrode 32, and a gate electrode 35 formed on an insulating layer 34 surrounding the emitter 33. By applying a predetermined extraction voltage between the cathode electrode 32 and the gate electrode 35, a high electric field is generated at the tip of the emitter 33, causing electrons to be emitted from the tip of the emitter 33.

Note that in the Spindt-type electron source, the hole penetrating through the gate electrode 35 and the insulating layer 34 is formed by etching, and the emitter 33 is made by depositing an emitter material inside the formed hole. The cold cathode electron source 30 may have a structure other than the Spindt-type structure. For example, the emitter 33 may be formed by a needle-like body made of a carbon nanotube or the like. Although not illustrated, one or more cold cathode electron sources 30 may be provided with one or more focus control electrodes to focus the electrons from the emitter 33.

Each of the electron emission units 12 shown in FIG. 3 is composed of a group of such cold cathode electron sources 30. The X-ray source 1 is equipped with a switching unit 17 for individually controlling the application of voltage to each of the electron emission units 12 (the group of cold cathode electron sources 30). The imaging control unit 6 (see FIG. 1) controls the power supply 8 to apply a predetermined voltage between the cathode electrode 32 (see FIG. 4) and the target 11. The imaging control unit 6 selectively connects the gate electrode 35 (see FIG. 4) of the cold cathode electron source 30 belonging to the selected electron emission unit 12 to the power supply 8 and controls the switching unit 17 to apply an extraction voltage to the gate electrode 35. As a result, an electron beam 36 is emitted from the group of the cold cathode electron sources 30 belonging to the selected electron emission unit 12, and X-rays 10 are emitted from the focal position 14 corresponding to the selected electron emission unit 12.

In the example shown in FIG. 3, one target 11 is provided for the plurality of electron emission units 12. The focal positions 14 of the plurality of electron emission units 12 are discretely positioned on the surface of the target 11, as shown in FIG. 5. In other words, the plurality of electron emission units 12 is formed so that the respective focal positions 14 are discretely distributed on the surface of the target 11. In FIG. 5, reflecting the array-like arrangement of the plurality of electron emission units 12 (see FIG. 3), focal positions 14 are arranged in an array on the surface of the target 11. The focal positions 14 are arranged at fixed distances 18a in the row direction. The focal positions 14 are arranged at fixed distances 18b in the column direction. The spot diameter (focal spot size) of the focal point formed by each individual electron emission unit 12 is smaller than the distances 18a and 18b between adjacent focal positions 14. This effectively reduces the focal spot size of the X-ray source 1. Further, the effect of heat generated by electron impingement on one of the focal positions 14 can be suppressed from spreading to the other adjacent focal positions 14.

### Switching Control of Electron Emission Units

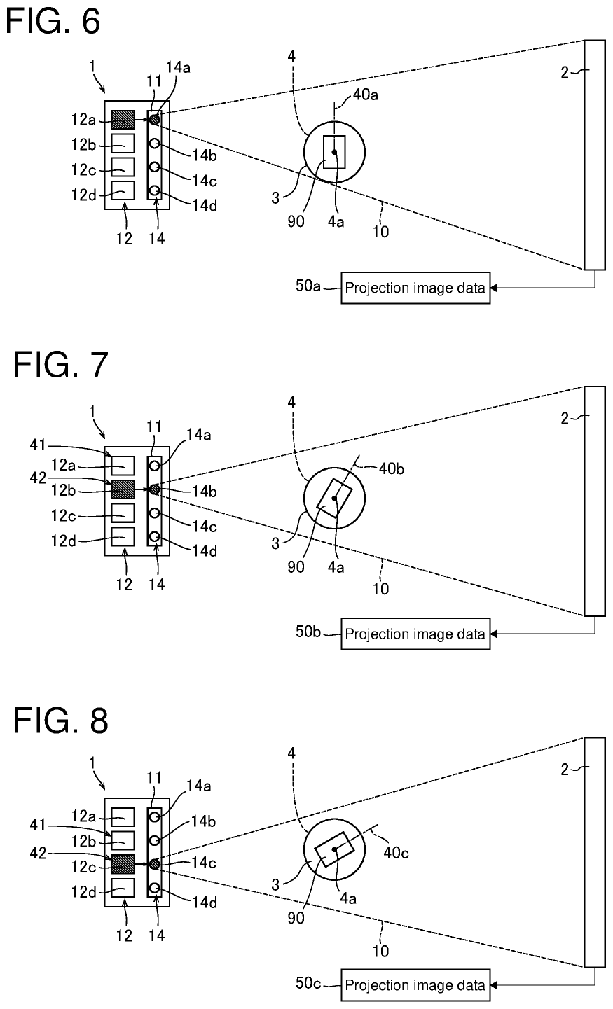

In this embodiment, the imaging control unit 6 (see FIG. 1) is configured to control the X-ray source 1 to perform X-ray irradiation by a subset of electron emission units 12 selected from the plurality of electron emission units 12 for each imaging angle 40 when acquiring the plurality of projection image data 50 (see FIG. 9), and also to control the selection of a second electron emission unit 42 (see FIGS. 6 to 8) different from the first electron emission unit 41 used for the immediately preceding X-ray irradiation (see FIGS. 7 and 8) when performing X-ray irradiation.

The imaging control unit 6 (see FIG. 1) controls the X-ray source 1 and the rotation mechanism 4 to acquire a plurality of projection image data 50 for each imaging angle 40 by repeating the acquisition of projection image data 50 by the X-ray source 1 and the detector 2 and the change of the imaging angle 40 by the rotation mechanism 4, as shown in FIG. 6 to FIG. 8. The imaging control unit 6 changes the electron emission unit 12 used for X-ray irradiation each time X-ray irradiation is performed to acquire projection image data 50. Note that in FIG. 6 to FIG. 8, the change range (unit angle) of the imaging angle 40 is enlarged for convenience of explanation.

Specifically, as shown in FIG. 6, the imaging control unit 6 initially selects any one of the electron emission units 12, e.g., the electron emission unit 12a, at the initial imaging angle 40a, and controls X-ray irradiation from the focal position 14a. The projection image data 50a at the imaging angle 40a is acquired by the detector 2. After acquiring the projection image data 50a, the imaging control unit 6 controls the rotation mechanism 4 to rotate the subject mounting unit 3 by a unit angle to change from the imaging angle 40a to the next imaging angle 40b (see FIG. 7).

As shown in FIG. 7, in the case of acquiring the projection image data 50b at the imaging angle 40b, the first electron emission unit 41 used for the immediately preceding X-ray irradiation is the electron emission unit 12a. The imaging control unit 6 selects an electron emission unit 12 different from the electron emission unit 12a, e.g., the electron emission unit 12b, as a second electron emission unit 42 different from the first electron emission unit 41. The imaging control unit 6 causes the selected electron emission unit 12b to perform X-ray irradiation from the focal position 14b and to acquire projection image data 50b at the imaging angle 40b. After acquiring the projection image data 50b, the imaging control unit 6 causes the imaging angle 40b to be changed to the next imaging angle 40c (see FIG. 8).

As shown in FIG. 8, in the case of acquiring the projection image data 50c at the imaging angle 40c, the first electron emission unit 41 used for the immediately preceding X-ray irradiation is the electron emission unit 12b. The imaging control unit 6 selects an electron emission unit 12 different from the electron emission unit 12b, e.g., the electron emission unit 12c, as a second electron emission unit 42 different from the first electron emission unit 41. The imaging control unit 6 causes the selected electron emission unit 12c to perform X-ray irradiation from the focal position 14c and to acquire projection image data 50c at the imaging angle 40c. After acquiring the projection image data 50c, the imaging control unit 6 changes from the imaging angle 40c to the next imaging angle 40.

The imaging control unit 6 (see FIG. 1) is configured to control the rotation mechanism 4 to be positioned at each of the plurality of imaging angles, which is defined by dividing 360 degrees by a preset number of imaging angles. Therefore, the imaging control unit 6 repeats the above-described control for the number of imaging angles to cause the imaging unit 7 to capture a plurality of projection image data 50 for 360 degrees.

With such a control, the electron emission unit 12 used for X-ray irradiation is changed each time X-ray irradiation is performed to acquire projection image data 50 for each of the imaging angles 40. With this control, as shown in FIG. 3, in the target 11 of the X-ray source 1, each time the projection image data 50 is acquired, another focal position 14, which is different from the focal position 14 where the X-ray irradiation was performed immediately before, is selected and the electron beam 36 is irradiated. With this, at the other focal position 14 selected this time, the effect of the heat generated by the collision of the electron beam 36 at the focal position 14 where the X-ray irradiation was performed immediately before is reduced. The heat generated by the collision of the electron beam 36 quickly diffuses in the target 11, thereby reducing the local heat load on the target 11 by the fact that the electron beam 36 is not continuously irradiated on the same single focal position 14.

Note that in the process of sequential imaging at each of the imaging angles 40, there may be some cases in which the same electron emission unit 12 used in the acquisition of the projection image data 50 until that time is selected. In this embodiment, it is sufficient to select a second electron emission unit 42 different from the first electron emission unit 41 used for the immediately preceding X-ray irradiation, and the same electron emission unit 12 that was used for the N^{th} (N is 2 or more) preceding X-ray irradiation may be selected for use in the current X-ray irradiation.

### Reconstruction Process

Next, the reconstruction process using a plurality of projection image data 50 (see FIG. 9) by the image processing unit 5 (see FIG. 1) will be described.

Initially, the data used for the reconstruction process will be described. As shown in FIG. 9, the storage unit 22 of the control device 20 (see FIG. 1) stores in advance the information 52 on the focal position 14 of each of the plurality of electron emission units 12. Each focal position 14 on the surface of the target 11 of the X-ray source 1 is determined based on the structural relation of the plurality of electron emission units 12 and the target 11 and is known. The information 52 on the focal position 14 is information capable of identifying at which position coordinates each of the focal positions 14 of the plurality of electron emission units 12 is located in the spatial coordinate system in the CT image reconstruction process. The information 52 on the focal position 14 is the coordinate (vector) information on the spatial coordinate system in the reconstruction processing program.

Note that the storage unit 22 stores programs 51 executed by the main control unit 21 and the image processing unit 5, as well as the setting information 53. The setting information 53 specifies the switching order of the plurality of electron emission units 12, the tube voltage, the number of imaging angles, and the constant data in the reconstruction process, etc. The respective projection image data 50 are stored in the storage unit 22 in association with the imaging angle 40 and the emission unit identification information 54 at the time the projection image data 50 were acquired. The emission unit identification information 54 is information for the electronic emission unit 12 used to acquire the projection image data 50 to identify which of the plurality of electron emission units 12. To generate one CT image 56, a set (hereinafter referred to as the “projection data set 55”) of projection image data 50 corresponding to the number of imaging angles set in the setting information 53 is stored in the storage unit 22. The storage unit 22 stores the generated CT images 56.

The image processing unit 5 (see FIG. 1) generates a CT image based on the projection data set 55 and the information 52 on the focal position 14 by executing the program 51 stored in the storage unit 22.

In this embodiment, the image processing unit 5 (see FIG. 1) is configured to generate a CT image by performing reconstruction process, including focal position correction of each of the plurality of projection image data 50, based on information 52 (see FIG. 9) on the focal position 14 of the electron emission unit 12 used to acquire each of the plurality of projection image data 50.

In this embodiment, as one example of the reconstruction process, a method for implementing focal position correction in the reconstruction process applying the FDK method (Feldkamp method), which is one type of analytical reconstruction methods, will be described.

As shown in FIG. 10, the spatial coordinate system in the reconstruction process is defined as Σ(x, y, z). Assuming the origin O (0, 0, 0) in the spatial coordinate system, the coordinates of the reconstruction target point are represented by the position vector r (x, y, z). Assuming that the rotation axis 4a passes through the origin O (0, 0, 0), the direction vector of the rotation axis 4a is (0, 0, 1). Each imaging angle 40 is represented by an angular variable β about the rotation axis 4a. Note that in this embodiment, the imaging unit 7 (the X-ray source 1 and the detector 2) is fixed, and the subject 90 (subject mounting unit 3) rotates, but for convenience, it is assumed in the following description that the imaging unit 7 (the X-ray source 1 and the detector 2) rotates about the rotation axis 4a. The rotation of the subject 90 about the rotation axis 4a and the rotation of the imaging unit 7 about the rotation axis 4a are equivalent in the reconstruction process.

Here, each projection image data 50 is obtained by X-rays emitted from any one of the focal positions 14 for each of the imaging angles 40. In FIG. 10, only four focal positions 14 are illustrated for convenience, but the focal positions 14 can be discretely distributed in an array-like manner, as shown in FIG. 5.

To correct the differences between the focal positions 14, a virtual rotation orbit 61 (shown by the dotted line) and a virtual focus 62 (shown by the black rectangular point) on the virtual rotation orbit 61 are assumed for each focal position. The virtual rotation orbit 61 is a virtual circular orbit representative of the orbit of each focal position 14 that rotates relative to the subject 90. Each focal position 14 is distributed on the surface of the target 11 and is not necessarily present on the virtual rotation orbit 61. The position of each focal position 14 can be expressed as a displacement from the virtual focus 62 on the virtual rotation orbit 61.

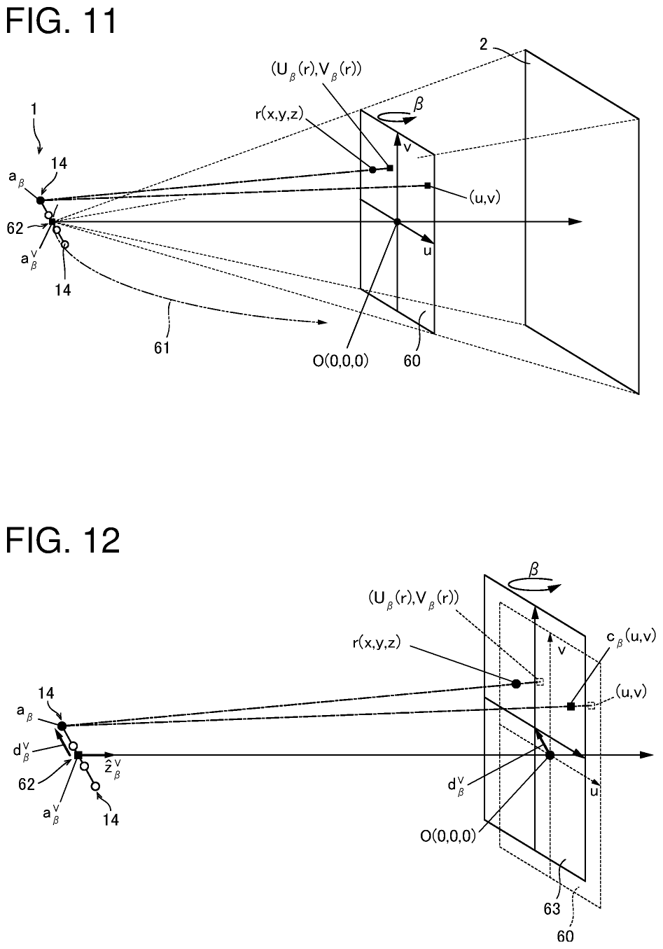

As shown in FIG. 11, a reference detection surface 60 is assumed with the detection surface of the detector 2 moved to the origin O (0, 0, 0). The origin of the reference detection surface 60 coincides with the origin O (0, 0, 0) of the spatial coordinate system, and the coordinates on the reference detection surface 60 are defined as (u, v). In FIG. 11 and FIG. 12, the focal position 14 at which X-ray irradiation is performed at a certain imaging angle β is indicated by a black circular dot, and the other focal positions 14 are indicated by white circular dots.

The reconstruction process according to this embodiment is roughly composed of three processing steps.
Processing Step 1: A weighting process is performed on each projection image data 50 at each imaging angle 40.
Processing Step 2: A filtering process is performed on each projection image data 50 at each imaging angle 40 after the weighting process.
Processing Step 3: A back-projection process is performed on the projection image data 50 for each of the imaging angles 40 after the filtering process.

### <Processing Step 1: Weighting Process>

The image processing unit 5 (see FIG. 1) is configured to perform a weighting process based on the information 52 on the focal position 14 corresponding to each of the plurality of projection image data 50, on each of the plurality of projection image data 50, in the reconstruction process.

The weighting process is performed by computing the following Formula (1).

$${g}^{W} ( β , u , v ) = \frac{‖ {a}_{β}^{V} ‖}{\sqrt{{‖ {a}_{β}^{V} ‖}^{2} + {‖ {c}_{β} ( u , v ) - {d}_{β}^{V} ‖}^{2}}} g ( β , u , v ) \qquad ( 1 )$$

$$where$$

$${d}_{β}^{V} = {a}_{β} - {a}_{β}^{V} \qquad ( 2 )$$

g is a projection value of the detection point (u, v) in the projection image data at the imaging angle β. g^{W} is a weighted projection value. a_{β} is a position vector of the actual focal position 14 at the imaging angle β. a_{β}^{V} is a position vector of the virtual focus 62 at the imaging angle B. As shown in FIG. 12, d_{β}^{V} is a vector that represents the displacement of the actual focal position 14 from the virtual focus 62. c_{β} is a position vector indicating the intersection coordinates of the straight line connecting the actual focal position a_{β} and the detection point (u, v) and the virtual detection surface 63. The actual focal position 14 and the virtual detection surface 63 are considered to be the parallel shift of the virtual focus 62 and the reference detection surface 60 by the displacement vector d_{β}^{V}.

The position vector a_{β}^{V} of the virtual focus 62 and the position vector c_{β} at the detection point on the virtual detection surface 63 are predetermined in the setting information 53 (program 51) and are known. The displacement vector d_{β}^{V} at the focal position is known from the information 52 on the focal position 14. The actual focal position vector a_{β} is represented by the position vector a_{β}^{V} at the virtual focus 62 and the displacement vector d_{β}^{V} at the focal position, as seen in Formula (2). The image processing unit 5 (see FIG. 1) acquires the values of these vectors, based on the information 52 on the focal position 14 and the setting information 53 (program 51) (see FIG. 9). Note that the information 52 on the focal position 14 only needs to include at least one of the position vector a_{β} of the actual focal position and the displacement vector d_{β}^{V} of the focal position.

As described above, in this embodiment, the image processing unit 5 (see FIG. 1) acquires the displacement d_{β}^{V} of the actual focal position aβ from the position a_{β}^{V} of the virtual focus 62, based on the information 52 of the focal position 14. By including the displacement d_{β}^{V} in Formula (1), the effect of the change of the focal position 14 for each imaging angle 40 in the weighting process is corrected.

### <Processing Step 2: Weighting Process>

The filtering process is performed by the calculation of the following Formula (3).

$${g}^{F} ( β , {u}^{′} , {v}^{′} ) = {\int }_{- {u}_{max}}^{{u}_{max}} h ( {u}^{′} - u ) {g}^{W} ( β , u , {v}^{′} ) du \qquad ( 3 )$$

where, g^{F} is a weighted projection value. h(u) is a reconstruction filter function. A known function, such as a Ram-Lak filter and a Shepp-Logan filter, is employed for the reconstruction filter function. Uₘₐₓ and −uₘₐₓ each represent the range of the filter direction in the projection image data.

### Processing Step 3: Back-Projection Processing

In this embodiment, the image processing unit 5 (see FIG. 1) is configured to perform a back-projection process based on the information 52 on the focal position 14 corresponding to each of the plurality of projection image data 50 for each of the plurality of projection image data 50, in the reconstruction process.

The back-projection process is performed by the calculation of the following Formula (4).

$$f ( r ) = \frac{1}{2} {\int }_{0}^{2 π} \frac{{‖ {a}_{β}^{V} ‖}^{2}}{{( ‖ {a}_{β}^{V} ‖ + ( r - {d}_{β}^{V} ) \cdot {\hat{z}}_{β}^{V} )}^{2}} {g}^{F} ( β , {U}_{β} ( r ) , {V}_{β} ( r ) ) d β \qquad ( 4 )$$

$$where$$

$${\hat{z}}_{β}^{V} = - {a}_{β}^{V} / ‖ {a}_{β}^{V} ‖ \qquad ( 5 )$$

where f(r) is a reconstructed value at the reconstruction target point r (x, y, z). By acquiring these reconstructed values over the entire subject 90, a CT image (each pixel value of the CT image) is obtained. U_{β}(r) and V_{β}(r) each represent the intersection coordinates (i.e., the detection point of the reconstruction target point r) of the straight line (i.e., the X-ray beam), which connects the actual focal position aβ and the reconstruction target point r, and the reference detection surface 60. z_{β}^{V} is the unit vector pointing from the virtual focus 62 to the origin, as shown in the above Formula (5). The image processing unit 5 (see FIG. 1) acquires the displacement vector d_{β}^{V} from the information 52 (see FIG. 9) on the focal position 14 and performs the back-projection process according to the above Formula (4).

As described above, in this embodiment, the image processing unit 5 (see FIG. 1) acquires the displacement d_{β}^{V} of the actual focal position aβ from the position a_{β}^{V} of the virtual focus 62 based on the information 52 on the focal position 14, and calculates a coefficient considering the displacement d_{β}^{V} in the above Formula (4), so that the effect of the change in the focal position 14 for each imaging angle 40 in the back-projection process is corrected.

As a result of the reconstruction process, the image processing unit 5 generates a CT image 56 (see FIG. 9) of the subject 90.

By the way, the virtual focus 62 and the virtual rotation orbit 61 correspond to a single focal position and its focal orbit when performing the reconstruction process on the projection image data acquired using a conventional single focal X-ray source. Therefore, if the reconstruction process premised on the conventional single focus is performed on a plurality of projection image data 50 acquired by selectively emitting X-rays 10 from a plurality of focal positions 14 as in this embodiment, an error corresponding to the displacement between the single focus and the actual focal position 14 (displacement vector d_{β}^{V}) will be included in each of the projection image data 50. In the reconstruction process of this embodiment described above, the actual focal position 14 (the electron emission unit 12) used to acquire the individual projection image data 50 is identified, and the displacement (the displacement vector d_{β}^{V}) between the virtual focus 62 and the actual focal position 14 is corrected, thereby suppressing the quality deterioration of the reconstructed image (CT image) due to the change in the focal position 14.

### Operation of X-ray Imaging Apparatus

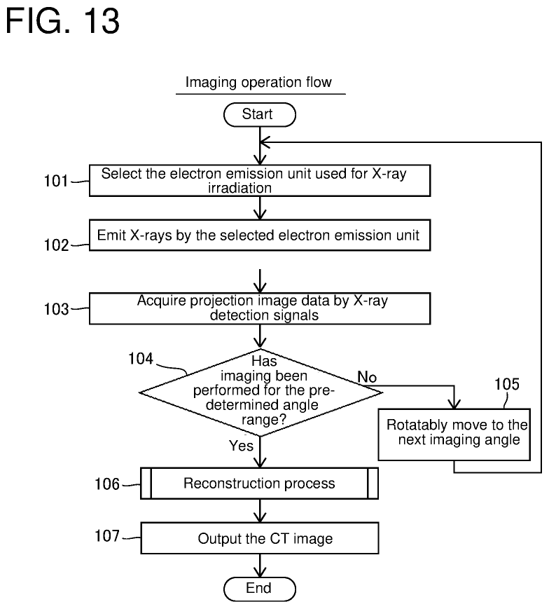

Next, referring to FIG. 13, the imaging operation of the X-ray imaging apparatus 100 of this embodiment will be described. The X-ray imaging apparatus 100 implements the X-ray imaging method according to this embodiment. The control of the imaging operation in the X-ray imaging apparatus 100 is performed by the imaging control unit 6. The acquisition of projection image data 50 and the generation of CT images are performed by the image processing unit 5. In the following description of operations, reference is made to FIG. 1 for the configuration of the X-ray imaging apparatus 100, and to FIG. 9 for various data.

The imaging operation is initiated when the control device 20 accepts an operational input via the input device 25. After a subject 90 is placed on the subject mounting unit 3, and an operation input to start imaging is received via the input device 25, the main control unit 21 transmits a signal to start the imaging operation to the imaging control unit 6. By receiving the signal from the main control unit 21, the imaging control unit 6 controls the X-ray source 1 and the rotation mechanism 4 to start the imaging operation of the subject 90.

In Step 101 of FIG. 13, the imaging control unit 6 selects the electron emission unit 12 to be used for X-ray irradiation at the current imaging angle 40 from among a plurality of electron emission units. At the time of the first imaging in the CT imaging, the electron emission unit 12 used for X-ray irradiation at the predetermined initial imaging angle 40 (β=0 degrees) is selected. The selection order has been preset in the setting information 53.

In Step 102, the imaging control unit 6 controls the X-ray source 1 to emit X-rays 10 using the electron emission unit 12 selected in Step 101. The imaging control unit 6 controls the switching unit 17 (see FIG. 3) to irradiate the target 11 with the electron beam 36 (see FIG. 3) from some electron emission units selected from among the plurality of electron emission units 12. With this, the X-rays 10 are emitted from the focal position 14 corresponding to the selected electron emission unit 12.

The X-rays 10 emitted from the focal position 14 of the X-ray source 1 pass through the subject 90 and are detected at the detector surface of the detector 2. In Step 103, the image processing unit 5 acquires (generates) the projection image data 50 from the image signals output by the detector 2. The acquired projection image data 50 is stored in the storage unit 22 in association with the imaging angle 40 and the emission unit identification information 54 of the used electron emission unit 12.

In Step 104, the imaging control unit 6 determines whether imaging (acquisition of projection image data 50) has been performed for the preset angular range (360 degrees). If the imaging for the preset angular range has not been executed, the imaging control unit 6 proceeds to Step 105.

In Step 105, the imaging control unit 6 controls the rotation mechanism 4 to move to the imaging angle 40 at which the next imaging is to be taken. The imaging control unit 6 controls the rotation mechanism 4 to rotate the subject mounting unit 3 and the imaging unit 7 relative to each other by a unit angle, which is obtained by dividing the preset angular range (360 degrees) by the number of imaging angles. In this embodiment, as described above, the rotation mechanism 4 rotates the subject mounting unit 3.

After that, the imaging control unit 6 returns the processing to Step 101. In Step 101, the imaging control unit 6 selects the electron emission unit 12 to be used for X-ray irradiation at the current imaging angle 40 from among the plurality of electron emission units 12. At this time, as shown in FIG. 7 and FIG. 8, the imaging control unit 6 selects a second electron emission unit 42 that is different from the first electron emission unit 41 used for the immediately preceding X-ray irradiation. Then, by Step 102 and Step 103, the projection image data 50 at the current imaging angle 40 are obtained.

The imaging control unit 6 repeats Step 101 to Step 105 the number of times corresponding to the number of imaging angles to capture each projection image data 50 within the preset angular range (360 degrees). As a result, the projection data set 55 composed of the projection image data 50 within the predetermined angular range (360 degrees) is stored in the storage unit 22. If imaging has been performed within the preset angular range, the imaging control unit 6 proceeds the process from Step 104 to Step 106.

In Step 106, the image processing unit 5 performs the above-described reconstruction process based on each projection image data 50 included in the projection data set 55.

In Step 107, the image processing unit 5 outputs a CT image 56 generated as a result of Step 106. The image processing unit 5 stores the CT image in the storage unit 22 and displays it on the display device 24. In this way, the imaging operation of the X-ray imaging apparatus 100 is performed.

As described above, the X-ray imaging method according to this embodiment comprises:

a first step (Step 102) of performing X-ray irradiation by a subset of electron emission units 12 selected from a plurality of electron emission units 12, from an X-ray source 1, the X-ray source 1 that includes a target 11 and a plurality of electron emission units 12, the X-ray source 1 being configured to emit electrons to different focal positions 14 on the target 11 such that electron beam axes from the plurality of electron emission units 12 do not intersect with each other;
a second step (Step 103) of acquiring projection image data 50 by detecting X-rays 10 emitted from the X-ray source 1 and transmitted through a subject 90 by a detector 2;
a third step (Step 105) of changing an imaging angle of the subject 90 by relatively rotating the X-ray source 1 and the detector 2 and the subject 90;
a fourth step (Step 101) of selecting a second electron emission unit 42 out of the plurality of electron emission units 12, the second electron emission unit 42 being different from the first electron emission unit 41 used for immediately preceding X-ray irradiation;
a step (Step 101 to Step 105) of acquiring a plurality of projection image data 50, one at each of imaging angles 40, by repeating the first step to the fourth step; and
a step (Step 106) of generating a CT image 56 based on the acquired plurality of projection image data 50.

### Control of Reconstruction Process

Next, the operation flow of the reconstruction process in Step 106 in FIG. 13 will be described.

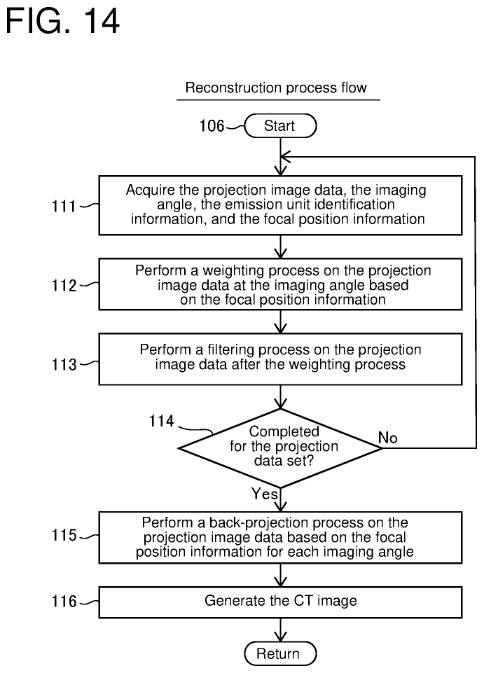

In Step 111 in FIG. 14, the image processing unit 5 acquires any of the projection image data 50 included in the projection data set 55 and the imaging angle 40 and the emission unit identification information 54 associated with the projection image data 50 from the storage unit 22. The image processing unit 5 acquires the information 52 (displacement vector dβV of the actual focal position 14 from the virtual focus 62) on the focal position 14 corresponding to the electron emission unit 12 identified by the emission unit identification information 54 from the storage unit 22.

In Step 112, the image processing unit 5 performs a weighting process by the above-described Formula (1) on the projection image data 50 at the imaging angle 40 based on the information 52 (displacement vector dβV) on the focal position 14.

In Step 113, the image processing unit 5 performs the filtering process by the above-described Formula (3) on the projection image data 50 after the weighting process.

In Step 114, the image processing unit 5 determines whether the weighting process and the filtering process have been completed for all of the multiple projection image data 50 included in the projection data set 55. In the case where the weighting process and the filtering process have not been completed for all of the projection image data 50, the image processing unit 5 returns the process to Step 111.

As a result of the repetition of Steps 111 to 114, the image processing unit 5 performs the weighting process and the filtering process on all of the projection image data 50 for each of the imaging angles 40 included in the projection data set 55. In this case, the image processing unit 5 proceeds to Step 115.

In Step 115, the image processing unit 5 performs a back-projection process by the above-described Formula (4) based on the information 52 on the focal position 14 (displacement vector d_{β}^{V}) on the projection image data 50 of each imaging angle 40 for which the weighting process and the filtering process have been performed.

As a result, in Step 116, the image processing unit 5 generates the CT image 56.

### Effect of This Embodiment

In this embodiment, the following effects can be achieved.

In this embodiment, as described above, the X-ray imaging apparatus 100 comprises:
an X-ray source 1 including a target 11 and a plurality of electron emission units 12, each of the plurality of electron emission units 12 being configured to emit electrons to different focal positions 14 on the target 11 such that the electron beam axes extending from each of the plurality of electron emission units 12 to the target do not intersect with each other;
a detector 2 configured to detect X-rays 10 emitted from the X-ray source 1;
a subject mounting unit 3 arranged between the X-ray source 1 and the detector 2 to support a subject 90;
a rotation mechanism 4 configured to relatively rotate an imaging unit 1 and the subject mounting unit 3 to change the imaging angle 40 of the subject 90, the imaging unit 7 including the X-ray source 1 and the detector 2;
an image processing unit 5 configured to acquire a plurality of projection image data 50, one at each of a plurality of the imaging angles 40, from the detector 2, and to generate a CT image 56 based on an acquired plurality of projection image data 50; and
an imaging control unit 6 configured to control the X-ray source 1 so that X-ray irradiation is performed by a subset of electron emission units 42 selected from the plurality of electron emission units 12, for each imaging angle 40 when acquiring the plurality of projection image data 50, and to control a selection of a second electron emission unit 42 different from the first electron emission unit 41 used in immediately preceding X-ray irradiation when performing X-ray irradiation.

Further, An X-ray imaging method comprising:
a first step (Step 102) of performing X-ray irradiation by a subset of electron emission units 12 selected from a plurality of electron emission units 12, from an X-ray source 1 that includes a target 11 and the plurality of electron emission units 12, the X-ray source 1 being configured to emit electrons to different focal positions 14 on the target 11 such that electron beam axes from the plurality of electron emission units 12 do not intersect with each other;
a second step (Step 103) of acquiring projection image data by detecting X-rays 10 emitted from the X-ray source 1 and transmitted through the subject 90 by a detector 2;
a third step (Step 105) of changing the imaging angle 40 of the subject 90 by relatively rotating the X-ray source 1 and the detector 2 and the subject 90;
a fourth step (Step 101) of selecting a second electron emission unit 42 out of the plurality of electron emission units 41, the second electron emission unit 42 being different from a first electron emission unit 41 used for immediately preceding X-ray irradiation;
a step (Step 101 to Step 105) of acquiring a plurality of projection image data 50, one at each of a plurality of imaging angles 40, by repeating the first step to the fourth step (Step 101); and
a step (Step 106) of generating a CT image 56 based on the acquired plurality of projection image data 50.

In this embodiment, with this, when sequentially performing X-ray irradiation for each imaging angle 40, it is possible to differentiate the focal position 14 on the target 11 in the immediately preceding X-ray irradiation by the first electron emission unit 41 and the focal position 14 on the target 11 in the subsequent X-ray irradiation by the second electron emission unit 42. Therefore, even if heat is generated intensively at the focal position 14 by reducing the focal spot size of X-rays, the focal position 14 on the target 11 is changed each time X-ray irradiation is performed, thereby distributing the heat-generating points on the target 11. As a result, compared with the case where the same focal position 14 of the target 11 continuously heats up, the local temperature rise on the target 11 can be reduced, thereby allowing damage to the target 11 to be suppressed even if the X-ray focal spot size is reduced.

Further, in this embodiment, the following additional effects can be obtained by configuring it as follows.

In other words, in this embodiment, as described above, the X-ray source 1 includes the electron source unit 15 having a plurality of cold cathode electron sources 30 arranged on a plane, and the plurality of electron emission units 12 are each composed of different groups of the plurality of cold cathode electron sources 30. With this, by grouping the plurality of cold cathode electron sources 30 arranged on a plane into individual groups, it is possible to collectively construct a plurality of electron emission units 12 that emit electrons to different focal positions 14 on the target 11. By using a micromachining technique, multiple (numerous) minute cold cathode electron sources 30 can be formed on the substrate surface simultaneously. Therefore, compared with a case in which a plurality of conventional thermal electron emission type electron sources is provided, a smaller size of the plurality of electron emission units 12 and a smaller focal spot size can be achieved. As a result, even in the case where the focal spot size formed by each electron emission unit 12 is reduced, it is possible to suppress damage to the target 11 caused by the heat generated by the collision of the electron beam 36 by the control of changing the focal position 14 on the target 11.

Further, in this embodiment, as described above, the group that constitutes one of the plurality of electron emission units 12 is configured by one or more cold cathode electron sources 30 that emit electrons to the same focal position 14 on the target 11. Thereby, since the electron emission unit 12 is configured by the cathode electron sources 30 arranged on a plane that emit electrons to the same focal position 14, among the plurality of cold cathode electron sources 30 arranged on a plane, it is possible to effectively reduce the focal spot size compared with the case where the electron emission unit 12 is configured by the plurality of cold cathode electron sources 30 dispersedly arranged on a plane.

Further, in this embodiment, as described above, one target 11 is provided for the plurality of electron emission units 12, and the focal positions 14 of each of the plurality of electron emission units 12 are discretely located on the surface of the target 11. With this, compared with the case in which a plurality of targets 11 is provided corresponding to a plurality of electron emission units 12, it is possible to simplify the configuration of the device. Further, since the focal positions 14 are discretely located on the surface of the target 11, even if heat is generated locally on the target 11 by electron beam irradiation to any one of the focal positions 14, the heat can be diffused while electron beam irradiation is performed to the other focal positions 14, thereby effectively reducing the heat load on the target 11.

Further, in this embodiment, as described above, it is further provided with the storage unit 22 that stores the information 52 on the focal position 14 of each of the plurality of electron emission units 12, and the image processing unit 5 is configured to generate a CT image 56 by performing the reconstruction process including the focal position correction of each of the plurality of projection image data 50, based on the information 52 on the focal position 14 of the electron emission units 12 used to acquire each of the plurality of projection image data 50. With this, even in the case where a plurality of electron emission units 12 that emit electrons at different focal positions 14 on the target 11 is provided, and each of the plurality of projection image data 50 is acquired while changing the focal position 14, it is possible to correct the effect of the change in the focal position 14 in the reconstruction process, based on the information 52 on the focal position 14. As a result, even in the case where each of the plurality of projection image data 50 is acquired using X-rays 10 emitted from different focal positions 14, it is possible to suppress the influence on the image quality of the CT image 56 caused by the change in the focal position 14.

Further, in this embodiment, as described above, the image processing unit 5 is configured to perform a weighting process based on the information 52 on the focal position 14 corresponding to each of the plurality of projection image data 50, on each of the plurality of projection image data 50, in the reconstruction process. With this, in the reconstruction process, it is possible to perform an appropriate weighting process considering the change in the focal position 14, for each of the plurality of projection image data 50. As a result, the effects such as the generation of artifacts caused by changes in the focal position 14 can be suppressed.

Further, in this embodiment, as described above, the image processing unit 5 is configured to perform a back-projection process based on the information 52 on the focal position 14 corresponding to each of the plurality of projection image data 50 for each of the plurality of projection image data 50, in the reconstruction process. With this, in the reconstruction process, it is possible to perform an appropriate back-projection process considering the change in the focal position 14, for each of the plurality of projection image data 50. As a result, the effects such as the generation of artifacts caused by changes in the focal position 14 can be suppressed.

Further, in this embodiment, as described above, the imaging control unit 6 is configured to control the rotation mechanism 4 so that it is positioned at each of a plurality of imaging angles 40, which are obtained by dividing 360 degrees by a preset number of imaging angles. With this configuration, by setting the number of imaging angles in the imaging control unit 6, it is possible to collect the projection image data 50 at arbitrary angular intervals (an arbitrary number of projection image data 50). With this, unlike the configuration in which, for example, a plurality of X-ray sources 1 is arranged to surround a subject 90, and CT imaging is performed at mechanically fixed constant angular intervals, it is possible to generate CT images 56 with appropriate spatial resolution according to the subject 90 and the purpose of the CT imaging.

### Modifications

Note that the embodiments disclosed here should be considered illustrative and not restrictive in all respects. It should be noted that the scope of the invention is indicated by claims and is intended to include all modifications (modified examples) within the meaning and scope of the claims and equivalents.

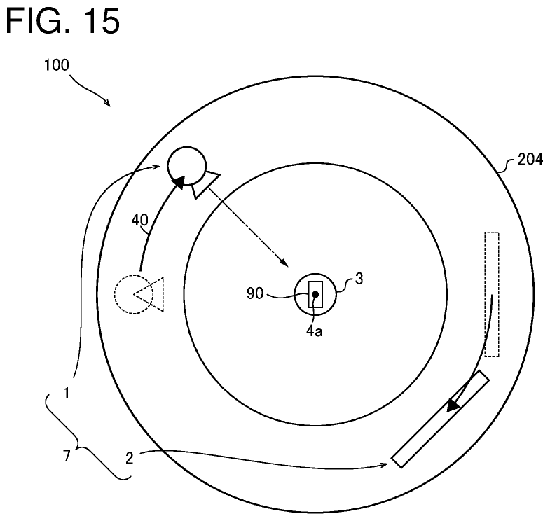

For example, in the above-described embodiment, an example is shown in which the rotation mechanism 4 rotates the subject mounting unit 3, thereby changing the imaging angle 40 of the subject 90, but the present invention is not limited to this. In the present invention, as shown in FIG. 15, the rotation mechanism 204 may rotate the imaging unit 7 (the X-ray source 1 and the detector 2), thereby changing the imaging angle 40 of the subject 90. In FIG. 15, the rotation mechanism 204 is configured to rotate the imaging unit 7, including the X-ray source 1 and the detector 2, about the rotation axis 4a. On the other hand, the subject mounting unit 3 is provided between the X-ray source 1 and the detector 2 in a non-rotatable manner. With this, by rotating the imaging unit 7, the imaging angle 40 of the subject 90 can be changed. Note that FIG. 15 shows the state in which the imaging unit 7 is rotated from the position indicated by the dashed line to the position indicated by the solid line.

Further, in the above-described embodiment, an example is shown in which one target 11 is provided for all of the plurality of electron emission units 12, but the present invention is not limited to this. The present invention may provide the same number of targets 11 as the plurality of electron emission units 12, and the electron emission units 12 and the targets 11 may be provided on a one-to-one basis. Further, one target 11 may be provided for some of the plurality of electron emission units 12. For example, in the case where it is assumed that there are electron emission units 12 in a 9×9 array, it may be configured such that one target 11 is provided for each group of 3×3, i.e., 9 electron emission units 12, thereby providing a total of 9 targets 11 for the 9×9 electron emission units.

Further, in the above-described embodiment, an example is shown in which the plurality of focal positions 14 is arranged in an array on the target 11 (see FIG. 5), but the present invention is not limited to this. In the present invention, the plurality of focal positions 14 may be arranged in a non-array manner. For example, the plurality of focal positions 14 may be arranged in a linear, concentric, or other patterns.

Further, in the above-described embodiment, an example is shown in which each of the plurality of imaging angles 40 is an angle defined by dividing 360 degrees by a preset number of imaging angles, but the present invention is not limited to this. The plurality of imaging angles 40 may be angles within a range greater than 360 degrees or less than 360 degrees. Further, in the above-described embodiment, the plurality of imaging angles 40 is set at equal angle intervals, but the plurality of imaging angles 40 may be set at non-equal angle intervals.

Further, in the above-described embodiment, an example is shown in which the image processing unit 5 performs the reconstruction process including the focal position correction of each of the plurality of projection image data 50, based on the information 52 on the focal position 14 of the electron emission unit 12 used to acquire each of the plurality of projection image data 50, but the present invention is not limited to this. In the present invention, a conventional reconstruction process that does not include a focal position correction may be performed. In such a case, as described above, a discrepancy will occur between the focal position (virtual focus 62) used for calculation and the actual focal position in each of the plurality of projection image data 50. Therefore, in order to improve the image quality of the CT image 56, it is preferable to perform a reconstruction process that includes a focal position correction.

Further, in the case of performing a reconstruction process including a focal position correction, the reconstruction process including the focal position correction shown in the above-described embodiment (Formulas (1) to (5)) is merely one example. The reconstruction process including the focal position correction according to the present invention is only required if the focal position correction is performed based on the information 52 on the focal position 14 of the electron emission unit 12 used to acquire each of the projection image data 50, and the calculation method (calculation formula) of the reconstruction process is not limited to the above-described Formulas (1) to (5).

Further, in the above-described embodiment, an example is shown in which in the reconstruction process, a weighting process (weighting process including the focal position correction) based on the information 52 on the focal position 14 and a back-projection process (back-projection process including the focal position correction) based on the information 52 on the focal position 14 are performed, but the present invention is not limited to this. In the present invention, for either the weighting process or the back-projection process, it is not necessary to perform a process based on the information 52 on the focal position 14 (focal position correction). In other words, for either the weighting process or the back-projection process, the displacement dβ of the focal position 14 from the virtual focus 62 need not be considered.

Further, in the above-described embodiment, as an example of the reconstruction process, an arithmetic method applying the FDK method, which is a kind of analytical method, is shown, but the present invention is not limited to this. In the present invention, as the reconstruction process of a CT image by the image processing unit 5, a reconstruction process by analytical methods other than the FDK method may be used. Further, a reconstruction process other than analytical methods, for example, using iterative methods, may also be used.

Further, in the above-described embodiment, an example is shown in which X-ray irradiation is performed using one electron emission unit 12 when acquiring projection image data 50 at one imaging angle 40, but the present invention is not limited to this. In the present invention, when acquiring projection image data 50 at one imaging angle 40, X-ray irradiation may be performed using two or more electron emission units (i.e., some of the plurality of electron emission units 12, and two or more of the plurality of electron emission units 12).

Further, in the above-described embodiment, an example of a device configuration in which X-rays 10 emitted from the X-ray source 1 directly pass through the subject 90 and enter the detector 2 is shown, but the present invention is not limited to this. In the present invention, X-ray interference imaging using the Talbot effect may be performed by placing a grating between the X-ray source 1 and the subject 90 and/or between the subject 90 and the detector 2. In other words, the X-ray imaging apparatus according to the present invention may be configured as a Talbot interferometer.

### Aspects

It would be understood by those skilled in the art that the exemplary embodiments described above are specific examples of the following aspects.

### (Item 1)

An X-ray imaging apparatus comprising:
an X-ray source including a target and a plurality of electron emission units, each of the plurality of electron emission units being configured to emit electrons to a different focal position on the target such that electron beam axes extending from the plurality of electron emission units to the target do not intersect with each other;
a detector configured to detect X-rays emitted from the X-ray source;
a subject mounting unit arranged between the X-ray source and the detector to support a subject;
a rotation mechanism configured to relatively rotate an imaging unit and the subject mounting unit to change an imaging angle of the subject, the imaging unit including the X-ray source and the detector;
an image processing unit configured to acquire a plurality of projection image data, one at each of a plurality of imaging angles, from the detector, and to generate a CT image based on an acquired plurality of projection image data; and
an imaging control unit configured to control the X-ray source so that X-ray irradiation is performed by a subset of electron emission units selected from the plurality of electron emission units, for each imaging angle when acquiring the plurality of projection image data, and to control a selection of a second electron emission unit different from a first electron emission unit used in immediately preceding X-ray irradiation when performing X-ray irradiation.

### (Item 2)

The X-ray imaging apparatus as recited in the above-described Item 1,
wherein the X-ray source includes an electron source unit having a plurality of cold cathode electron sources arranged on a plane, and
wherein the plurality of electron emission units is each composed of mutually different groups of the plurality of cold cathode electron sources.

### (Item 3)

The X-ray imaging apparatus as recited in the above-described Item 2,
wherein the group constituting one of the plurality of electron emission units is composed of one or more cold cathode electron sources that emit electrons to the same focal position of the target.

### (Item 4)

The X-ray imaging apparatus as recited in the above-described Item 1,
wherein the target is provided as a single target for the plurality of electron emission units, and
wherein the focal positions of the plurality of electron emission units are discretely positioned on a surface of the target.

### (Item 5)

The X-ray imaging apparatus as recited in the above-described Item 1, further comprising:
a storage unit configured to store information on the focal position of each of the plurality of electron emission units,
wherein the image processing unit is configured to generate the CT image by performing a reconstruction process, including focal position correction of each of the plurality of projection image data, based on information on the focal position of the electron emission unit used to acquire each of the plurality of projection image data.

### (Item 6)

The X-ray imaging apparatus as recited in the above-described Item 5,
wherein the image processing unit is configured to perform a weighting process on each of the plurality of projection image data in the reconstruction process, the weighting process being based on the information on the focal position corresponding to each of the plurality of projection image data.

### (Item 7)

The X-ray imaging apparatus as recited in the above-described Item 5,
wherein the image processing unit is configured to perform a back-projection process on each of the plurality of projection image data in the reconstruction process, the back-projection process being based on information on the focal position corresponding to each of the plurality of projection image data.

### (Item 8)

The X-ray imaging apparatus as recited in the above-described Item 1,
wherein the imaging control unit is configured to control the rotation mechanism so that the rotation mechanism is positioned at each of a plurality of imaging angles defined by dividing 360 degrees by a pre-set number of imaging angles.

### (Item 9)

An X-ray imaging method comprising:
a first step of performing X-ray irradiation by a subset of electron emission units selected from a plurality of electron emission units, from an X-ray source that includes a target and the plurality of electron emission units, the X-ray source being configured to emit electrons to different focal positions on the target such that electron beam axes extending from the plurality of electron emission units to the target do not intersect with each other;
a second step of acquiring projection image data by detecting X-rays emitted from the X-ray source and transmitted through a subject by a detector;
a third step of changing an imaging angle of the subject by relatively rotating the X-ray source and the detector and the subject;
a fourth step of selecting a second electron emission unit out of the plurality of electron emission units, the second electron emission unit being different from a first electron emission unit used for immediately preceding X-ray irradiation;
a step of acquiring a plurality of projection image data, one at each of a plurality of imaging angles, by repeating the first step to the fourth step; and
a step of generating a CT image based on the acquired plurality of projection image data.

### DESCRIPTION OF REFERENCE SYMBOLS

1: X-ray source
2: Detector
3: Subject mounting unit
4, 204: Rotation mechanism
5: Image processing unit
6: Imaging control unit
7: Imaging unit
10: X-rays
11: Target
12 (12a, 12b, 12c, 12d): Electron emission unit
14 (14a, 14b, 14c, 14d): Focal position
15: Electron source unit
22: Storage unit
30: Cold cathode electron source
40 (40a, 40b, 40c): Imaging angle
41: First electron emission unit
42: Second electron emission unit
50 (50a, 50b, 50c): Projection image data
52: Information on focal position
56: CT image
90: Subject
100: X-ray imaging apparatus

1. An X-ray imaging apparatus comprising:
an X-ray source including a target and a plurality of electron emission units, each of the plurality of electron emission units being configured to emit electrons to a different focal position on the target such that electron beam axes extending from the plurality of electron emission units to the target do not intersect with each other;
a detector configured to detect X-rays emitted from the X-ray source;
a subject mounting unit arranged between the X-ray source and the detector to support a subject;
a rotation mechanism configured to relatively rotate an imaging unit and the subject mounting unit to change an imaging angle of the subject, the imaging unit including the X-ray source and the detector;
an image processing unit configured to acquire a plurality of projection image data, one at each of a plurality of imaging angles, from the detector, and to generate a CT image based on an acquired plurality of projection image data; and
an imaging control unit configured to control the X-ray source so that X-ray irradiation is performed by a subset of electron emission units selected from the plurality of electron emission units, for each imaging angle when acquiring the plurality of projection image data, and to control a selection of a second electron emission unit different from a first electron emission unit used in immediately preceding X-ray irradiation when performing X-ray irradiation.

2. The X-ray imaging apparatus as recited in claim 1,
wherein the X-ray source includes an electron source unit having a plurality of cold cathode electron sources arranged on a plane, and
wherein the plurality of electron emission units is each composed of mutually different groups of the plurality of cold cathode electron sources.

3. The X-ray imaging apparatus as recited in claim 2,
wherein the group constituting one of the plurality of electron emission units is composed of one or more cold cathode electron sources that emit electrons to the same focal position of the target.

4. The X-ray imaging apparatus as recited in claim 1,
wherein the target is provided as a single target for the plurality of electron emission units, and
wherein the focal positions of the plurality of electron emission units are discretely positioned on a surface of the target.

5. The X-ray imaging apparatus as recited in claim 1, further comprising:
a storage unit configured to store information on the focal position of each of the plurality of electron emission units,
wherein the image processing unit is configured to generate the CT image by performing a reconstruction process, including focal position correction of each of the plurality of projection image data, based on information on the focal position of the electron emission unit used to acquire each of the plurality of projection image data.

6. The X-ray imaging apparatus as recited in claim 5,
wherein the image processing unit is configured to perform a weighting process on each of the plurality of projection image data in the reconstruction process, the weighting process being based on the information on the focal position corresponding to each of the plurality of projection image data.

7. The X-ray imaging apparatus as recited in claim 5,
wherein the image processing unit is configured to perform a back-projection process on each of the plurality of projection image data in the reconstruction process, the back-projection process being based on information on the focal position corresponding to each of the plurality of projection image data.

8. The X-ray imaging apparatus as recited in claim 1,
wherein the imaging control unit is configured to control the rotation mechanism so that the rotation mechanism is positioned at each of a plurality of imaging angles defined by dividing 360 degrees by a pre-set number of imaging angles.

9. An X-ray imaging method comprising:
a first step of performing X-ray irradiation by a subset of electron emission units selected from a plurality of electron emission units, from an X-ray source that includes a target and the plurality of electron emission units, the X-ray source being configured to emit electrons to different focal positions on the target such that electron beam axes extending from the plurality of electron emission units to the target do not intersect with each other;
a second step of acquiring projection image data by detecting X-rays emitted from the X-ray source and transmitted through a subject by a detector;
a third step of changing an imaging angle of the subject by relatively rotating the X-ray source and the detector and the subject;
a fourth step of selecting a second electron emission unit out of the plurality of electron emission units, the second electron emission unit being different from a first electron emission unit used for immediately preceding X-ray irradiation;
a step of acquiring a plurality of projection image data, one at each of a plurality of imaging angles, by repeating the first step to the fourth step; and
a step of generating a CT image based on the acquired plurality of projection image data.
